(19) United States
(12) Patent Application Publication
(10) Pub. No.: US 2004/0148001 A1
(43) Pub. Date: Jul. 29, 2004

(54) Solvent-bonded stent-graft assembly

(76) Inventor: John E. Nolting, Santa Rosa, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/350,821

(22) Filed: Jan. 24, 2003

(51) Int. Cl. A61F 2/06

(52) U.S. Cl. 623/1.11; 623/1.42; 623/1.13

(57) ABSTRACT

The present invention provides a solvent-bonded stent-graft assembly. A solvatable polymer coating is applied to a stent framework. A generally cylindrical graft member is placed adjacent to the solvatable polymer coating, and this assembly is subjected to a solvent. The solvatable polymer coating is conjoined with the graft member using the resulting solvate.

### TECHNICAL FIELD

This invention relates generally to biomedical devices that are used for treating vascular conditions. More specifically, the invention relates to using solvent-based bonding to conjoin a stent and a generally cylindrical graft member to form a stent-graft assembly.

### BACKGROUND OF THE INVENTION

Stents are generally cylindrical-shaped devices that are radially expandable to hold open a segment of a vessel or other anatomical lumen after implantation into the body lumen.

Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guide wire that is part of the delivery device. After the stent is positioned across the lesion, it is expanded by the delivery device, causing the length of the stent to contract and the diameter to expand. For a self-expanding stent, commonly a sheath is retracted, allowing expansion of the stent.

Stents are used in conjunction with balloon catheters in a variety of medical therapeutic applications, including intravascular angioplasty. For example, a balloon catheter device is inflated during percutaneous transluminal coronary angioplasty (PTCA) to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter facilitates improved blood flow. Soon after the procedure, however, a significant proportion of treated vessels restenose.

To prevent restenosis, stents, constructed of metal or various polymers, are implanted within the vessel to maintain lumen size. The stent acts as a scaffold to support the lumen in an open position. Configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau.

Stent insertion may cause undesirable reactions such as inflammation, infection, thrombosis, and proliferation of cell growth that occludes the passageway. Stents have been used with coatings to deliver drugs or other therapeutic agents at the site of the stent that may assist in preventing these conditions. In some methods of producing a stent designed to deliver a drug, the drug coating is applied to a stent framework. This may result in drug being delivered to only those portions of the vessel in direct contact with the stent, providing as little as 20% coverage.

Stents may be used alone or in conjunction with a graft. The graft component of a stent-graft may aid in minimizing thrombosis, preventing embolic events, and minimizing contact between the fissured plaque and the hematological elements in the bloodstream. It may also be used to deliver drugs or other therapeutic agents for medical therapeutic applications.

In addition, the graft component makes the device suitable for use in treating aneurysms. An aneurysm is a bulge or sac that forms in the wall of a blood vessel. The force of normal blood pressure in the aneurysm may cause the vessel to rupture. Aneurysms are most commonly the result of fatty deposits on the vessel wall but may also result from other causes that weaken the vessel wall, including heredity, trauma, or disease.

A number of methods and devices have been developed for treating aneurisms. A standard treatment is conventional open surgery, which is performed to replace the section of the vessel where the aneurysm has formed. Some patients are not good candidates for such open surgery, and, due to the highly invasive nature of the open procedure, other patients may not wish to undergo the treatment.

An alternative treatment is a technique known as endovascular stent grafting. In this procedure, a stent-graft is placed inside the vessel affected by the aneurysm in order to reinforce the weakened vessel wall, thereby preventing rupture of the aneurysm. Like stents, stent-grafts are delivered to the area to be treated using balloon catheters or other expandable devices Various methods have been devised to produce stent-grafts. Some of these, such as methods that involve stitching the graft material to the stent, can be labor intensive. Other methods may provide inadequate adherence of the graft material to the stent or a delivery profile that is larger than would be ideal.

Therefore, it would be desirable to have a stent-graft assembly that overcomes the aforementioned and other disadvantages.

### SUMMARY OF THE INVENTION

One aspect of the present invention is a system for treating a vascular condition, comprising a catheter, a stent operably coupled to the catheter, the stent including a stent framework; a solvatable polymer coating disposed on at least a portion of the stent framework; and a generally cylindrical graft member, wherein the graft member is conjoined with the solvatable polymer coating using a solvent.

Another aspect of the present invention is a stent-graft assembly, comprising a stent framework; a solvatable polymer coating disposed on at least a portion of the stent framework; and a generally cylindrical graft member, wherein the graft member is conjoined with the solvatable polymer coating using a solvent.

A further aspect of the present invention is a method of manufacturing a stent-graft assembly. A stent framework is provided. A solvatable polymer coating is applied to at least a portion of the stent framework. A generally cylindrical graft member is placed adjacent to the solvatable polymer coating. The solvatable polymer coating and adjacent graft member are subjected to a solvent. The solvatable polymer coating is conjoined with the graft member using the resulting solvate. The conjoined solvatable polymer coating and graft member are allowed to solidify.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a system for treating a vascular condition, in accordance with the present invention;

FIG. 2 is a cross-section of one embodiment of a stent-graft assembly, in accordance with the present invention;

FIG. 3 is a partial cross-section of one embodiment of a stent-graft assembly shown before it has been subjected to a solvent, in accordance with the present invention;

FIG. 4 is a partial cross-section of one embodiment of a stent-graft assembly shown after it has been subjected to a solvent, in accordance with the present invention;

FIG. 5 is a partial cross-section of one embodiment of a stent-graft assembly shown before it has been subjected to a solvent, in accordance with the present invention;

FIG. 6 is a partial cross-section of one embodiment of a stent-graft assembly shown after it has been subjected to a solvent, in accordance with the present invention;

FIG. 7 is a flow diagram of one embodiment of a method of manufacturing a stent-graft assembly, in accordance with the present invention.

### DETAILED DESCRIPTION OF THE PRESENTLY PREFERRED EMBODIMENTS

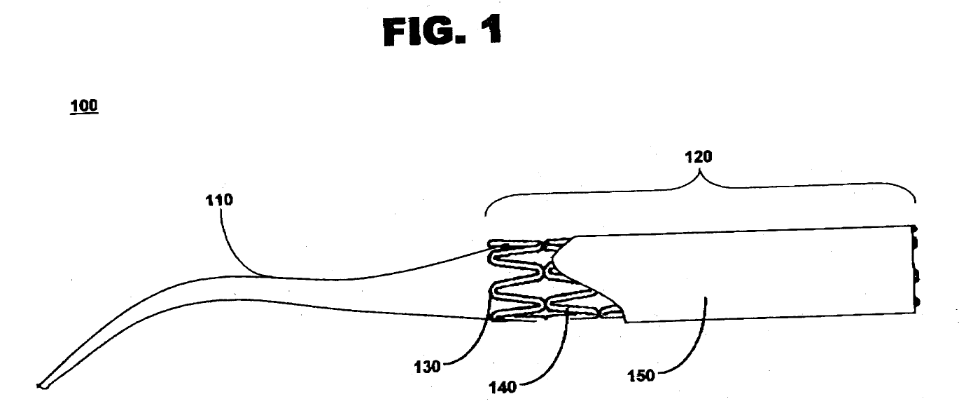

One aspect of the present invention is a system for treating a vascular condition. One embodiment of the system, in accordance with the present invention, is illustrated in FIG. 1 at 100. System 100 comprises a catheter 110 and a stent 120 operably coupled to the catheter. Stent 120 includes a stent framework 130. At least a portion of the stent framework is coated with a solvatable polymer coating 140. Through the term solvatable, I am referring to a polymer coating which is capable of being dissolved, at least partially, by a solvent. Of course, the exact degree to which the polymer may be dissolved varies according to the polymer itself and the corresponding solvent used. A generally cylindrical graft member 150 is conjoined with the solvatable polymer coating using a solvent. Graft member 150 may be either external, as shown, or internal to stent framework 130. Graft member 150 is shown partially cut away to reveal the stent framework below.

Catheter 110 may include a balloon to expand the stent, or it may include a sheath that retracts to allow expansion of a self-expanding stent. Both types of catheter are well known in the art. Stent 120 is shown coupled to catheter 110 for delivery within a vessel.

Stent framework 130 may be made of a wide variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above.

Solvatable polymer coating 140 may comprise, for example, ChronoFlex® AR polyurethane, other polyurethanes, fluorinated ethylene propylene (FEP), tetrafluoroethylene, polytetrafluoroethylene (PTFE), combinations of the above, and the like. The coating may include a therapeutic agent such as an antineoplastic agent, an antiproliferative agent, an antibiotic, an antithrombogenic agent, an anticoagulant, an antiplatelet agent, an anti-inflammatory agent, combinations of the above, and the like.

Graft member 150 may comprise a solvatable polymer. The solvatable polymer comprising graft member 150 may be the same as that comprising the solvatable polymer coating. Alternatively, the graft member may comprise a porous material different from that comprising the solvatable polymer coating. The porous material may be, for example, polytetrafluoroethylene (PTFE). Graft member 150 may include a therapeutic agent such as an antineoplastic agent, an antiproliferative agent, an antibiotic, an antithrombogenic agent, an anticoagulant, an antiplatelet agent, an anti-inflammatory agent, combinations of the above, and the like.

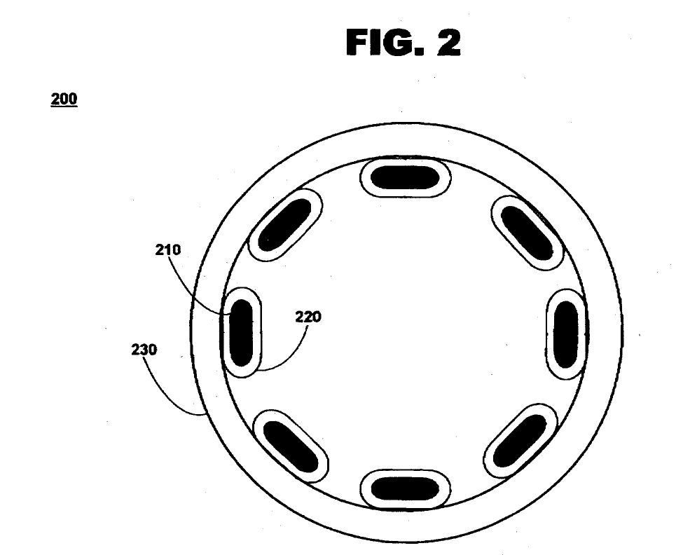

Another aspect of the present invention is a stent-graft assembly. A cross-section of one embodiment of the assembly, in accordance with the present invention, is illustrated in FIG. 2 at 200. Assembly 200 includes a stent framework 210. A solvatable polymer coating 220 is disposed on at least a portion of the stent framework. A generally cylindrical graft member 230 may be either external, as shown, or internal to stent framework 210. To most easily distinguish the separate elements, the assembly is shown prior to the graft member being conjoined with the solvatable polymer coating using a solvent.

Stent framework 210 may be made of a wide variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above.

Solvatable polymer coating 220 may comprise, for example, ChronoFlex® AR polyurethane, other polyurethanes, fluorinated ethylene propylene (FEP), tetrafluoroethylene, polytetrafluoroethylene (PTFE), combinations of the above, and the like. The coating may include a therapeutic agent such as an antineoplastic agent, an antiproliferative agent, an antibiotic, an antithrombogenic agent, an anticoagulant, an antiplatelet agent, an anti-inflammatory agent, combinations of the above, and the like.

Graft member 230 may comprise a solvatable polymer. The solvatable polymer comprising graft member 230 may be the same as that comprising the solvatable polymer coating. Alternatively, the graft member may comprise a porous material different from that comprising the solvatable polymer coating. The porous material may be, for example, polytetrafluoroethylene (PTFE). Graft member 230 may include a therapeutic agent such as an antineoplastic agent, an antiproliferative agent, an antibiotic, an antithrombogenic agent, an anticoagulant, an antiplatelet agent, an anti-inflammatory agent, combinations of the above, and the like.

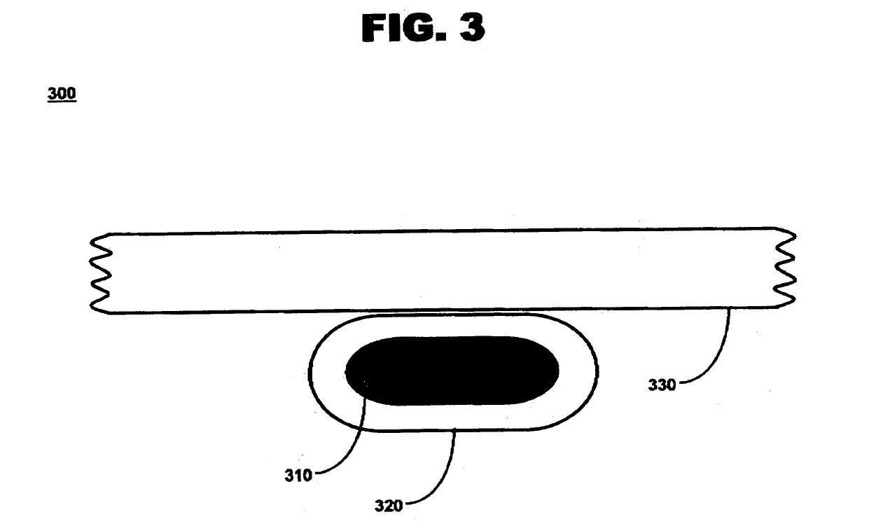

A partial cross-section of one embodiment of a stent-graft assembly, in accordance with the present invention, is illustrated in FIG. 3 at 300. The assembly is shown before it has been subjected to a solvent. In this embodiment, the coating and the graft member comprise the same solvatable polymer. A single wire or other element of a stent framework 310 is shown coated with a solvatable polymer coating 320 and adjacent to a portion of a graft member 330.

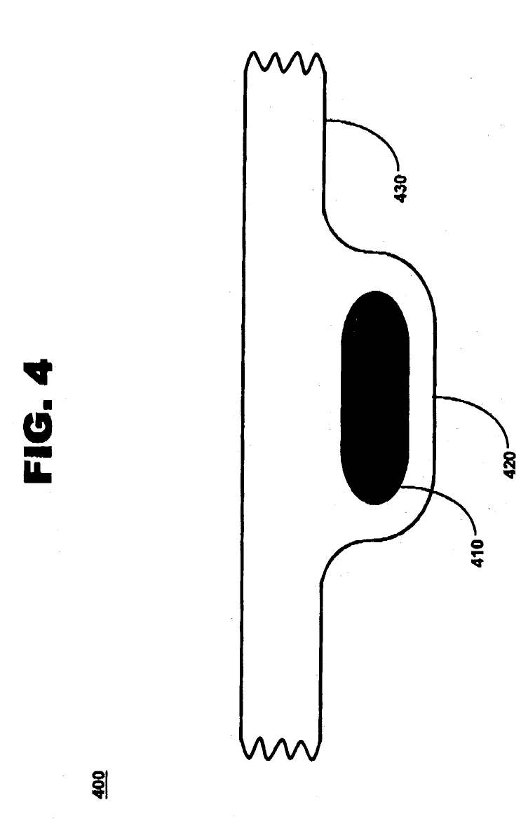

A partial cross-section of the same embodiment of a stent-graft assembly that was shown in FIG. 3 is illustrated in FIG. 4 at 400 after the assembly has been subjected to a solvent. The solvent has at least partially dissolved both the coating and the graft member, which comprise the same solvatable polymer, allowing them to meld into a unitary structure. A single wire or other element of a stent framework 410 is shown coated with a solvatable polymer coating 420 that has been conjoined with a portion of a graft member 430.

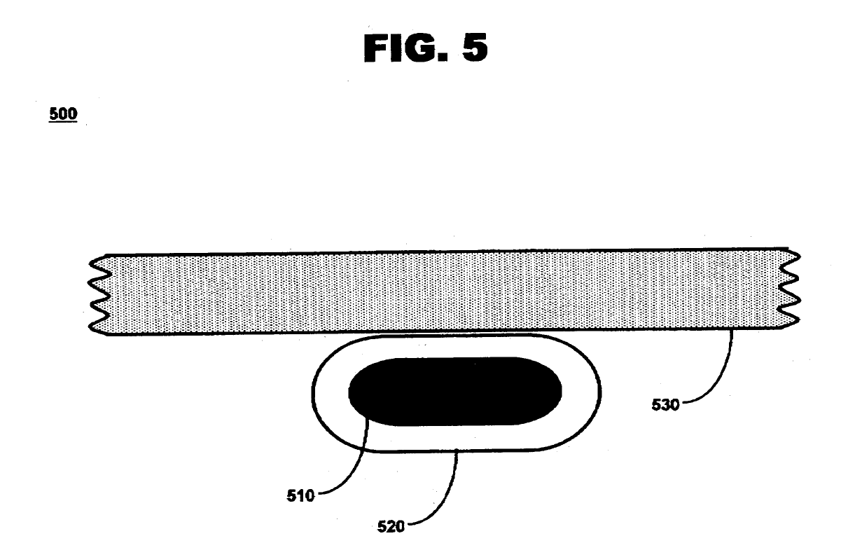

A partial cross-section of another embodiment of a stent-graft assembly, in accordance with the present invention, is illustrated in FIG. 5 at 500. The assembly is shown before it has been subjected to a solvent. In this embodiment, the graft member comprises a porous material that is different from the material comprising the solvatable polymer coating. A single wire or other element of a stent framework 510 is shown coated with a solvatable polymer coating 520 and adjacent to a portion of a graft member 530.

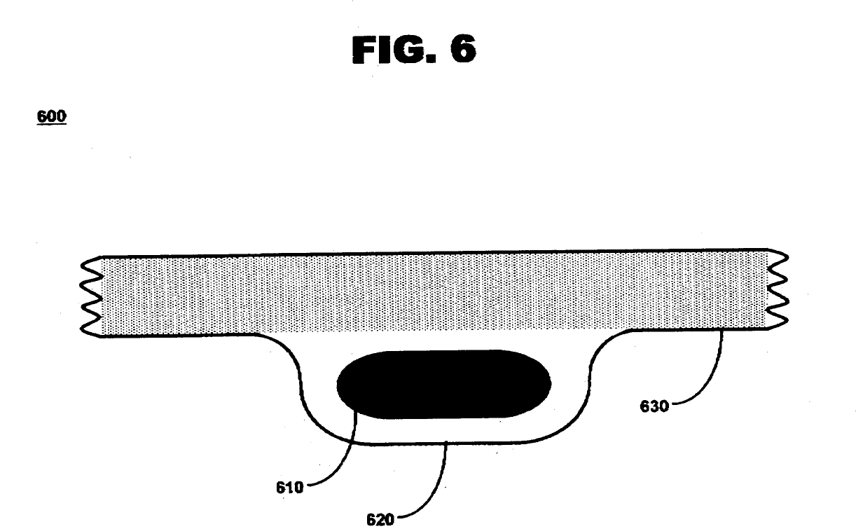

A partial cross-section of the same embodiment of a stent-graft assembly that was shown in FIG. 5 is illustrated in FIG. 6 at 600 after the assembly has been subjected to a solvent. In this illustration, the graft member again comprises a porous material that is different from the material comprising the solvatable polymer coating. The solvent has at least partially dissolved the coating, allowing it to flow into the pores of the graft member, thereby adhering the graft member to the coating. A single wire or other element of a stent framework 610 is shown coated with a solvatable polymer coating 620 that has been conjoined with a portion of a graft member 630.

A further aspect of the present invention is a method of manufacturing a stent-graft assembly. FIG. 7 shows a flow diagram of one embodiment, in accordance with the present invention at 700.

In this embodiment, a stent framework is provided (Block 710). The stent framework may be made of a wide variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above.

A solvatable polymer coating is applied to the stent framework (Block 720). The coating may comprise, for example, ChronoFlex® AR polyurethane or another suitable polymer and may include a therapeutic agent. The coating may be applied by a method such as pad printing, inkjet printing, rolling, painting, spraying, micro-spraying, dipping, wiping, electrostatic deposition, vapor deposition, epitaxial growth, and combinations thereof.

A generally cylindrical graft member is placed adjacent to the stent framework (Block 730). The graft member may comprise a solvatable polymer that is the same as that comprising the coating. Alternatively, the graft member may comprise a porous material that is different from the material comprising the coating. The porous material may be, for example, polytetrafluoroethylene (PTFE). The graft member may include a therapeutic agent that has been incorporated within or applied to the material comprising the graft.

The cylindrical graft member may be created by, for example, forming a graft material into a generally cylindrical shape around the stent framework, extruding a generally cylindrical graft member and placing it within or without the stent framework, forming the graft material onto a mandrel, or any method of producing a generally cylindrical graft member known in the art. The stent framework may be expanded within the graft member or compressed about the graft member to place it adjacent to the stent framework.

The solvatable polymer coating and adjacent graft member are subjected to a solvent (Block 740). This may be accomplished by placing the assembly in a saturated atmosphere of a solvent vapor, dipping the assembly into a liquid solvent, spraying the assembly with a liquid or vapor solvent, or any method that would at least partially solvate the coating alone or both the coating and the graft member. The solvent may be, for example, N,N-dimethylacetamide (DMAC) or another appropriate solvents, such as THF, chloroform, cyclohexane, methanol, ethanol, methylene chloride to name only a few.

The solvatable polymer coating and adjacent graft member are conjoined using the resulting solvate (Block 750). For example, where the coating and the graft member comprise the same material, the solvated coating and graft material may meld into a unitary structure. Alternatively, where the coating and the graft member comprise different materials, the solvated coating may flow into the pores of the graft member, thereby adhering the graft member to the coating.

The conjoined coating and graft member are then solidified (Block 760). This may be accomplished by heating, air-drying, curing, combinations thereof, and the like.

In practice, the present invention provides a method of securely attaching a graft material to a coated stent. The resulting stent-graft is not only durable, but has the further advantage of a small delivery profile.

While the embodiments of the invention disclosed herein are presently considered preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for treating a vascular condition, comprising:
a catheter;
a stent operably coupled to the catheter, the stent including a stent framework;
a solvatable polymer coating disposed on at least a portion of the stent framework; and
a generally cylindrical graft member, wherein the graft member is conjoined with the solvatable polymer coating using a solvent.

2. The system of claim 1 wherein the graft member comprises a solvatable polymer.

3. The system of claim 2 wherein the solvatable polymer comprising the coating and the solvatable polymer comprising the graft member are the same solvatable polymer.

4. The system of claim 1 wherein the graft member comprises a porous material different from the material comprising the solvatable polymer coating.

5. The system of claim 1 wherein the solvatable polymer coating includes a therapeutic agent selected from a group consisting of an antineoplastic agent, an antiproliferative agent, an antibiotic, an antithrombogenic agent, an anticoagulant, an antiplatelet agent, and an anti-inflammatory agent.

6. The system of claim 1 wherein the graft member includes a therapeutic agent selected from a group consisting of an antineoplastic agent, an antiproliferative agent, an antibiotic, an antithrombogenic agent, an anticoagulant, an antiplatelet agent, and an anti-inflammatory agent.

7. The system of claim 1 wherein the graft member is external to the stent framework.

8. The system of claim 1 wherein the graft member is internal to the stent framework.

9. The system of claim 1 wherein the catheter includes a balloon used to expand the stent.

10. The system of claim 1 wherein the catheter includes a sheath that retracts to allow expansion of the stent.

11. A stent-graft assembly, comprising:
a stent framework;
a solvatable polymer coating disposed on at least a portion of the stent framework; and
a generally cylindrical graft member, wherein the graft member is conjoined with the solvatable polymer coating using a solvent.

12. The assembly of claim 11 wherein the polymer comprising the graft member is a solvatable polymer.

13. The assembly of claim 12 wherein the solvatable polymer comprising the coating and the solvatable polymer comprising the graft member are the same solvatable polymer.

14. The assembly of claim 11 wherein the graft member comprises a porous material different from the material comprising the solvatable polymer coating.

15. The assembly of claim 10 wherein the graft member includes a therapeutic agent selected from a group consisting of an antineoplastic agent, an antiproliferative agent, an antibiotic, an antithrombogenic agent, an anticoagulant, an antiplatelet agent, and an anti-inflammatory agent.

16. The assembly of claim 11 wherein the solvatable polymer coating includes a therapeutic agent selected from a group consisting of an antineoplastic agent, an antiproliferative agent, an antibiotic, an antithrombogenic agent, an anticoagulant, an antiplatelet agent, and an anti-inflammatory agent.

17. The assembly of claim 11 wherein the graft member is external to the stent framework.

18. The assembly of claim 11 wherein the graft member is internal to the stent framework.

19. A method of manufacturing a stent-graft assembly, comprising:
providing a stent framework;
applying a solvatable polymer coating to at least a portion of the stent framework;
placing a generally cylindrical graft member adjacent to the solvatable polymer coating;
subjecting the solvatable polymer coating and adjacent graft member to a solvent;
conjoining the solvatable polymer coating with the graft member using the resulting solvate; and
solidifying the conjoined solvatable polymer coating and graft member.

20. The method of claim 19 wherein the solvatable polymer coating is applied by a method selected from the group consisting of pad printing, ink-jet printing, rolling, painting, spraying, micro-spraying, dipping, wiping, electrostatic deposition, vapor deposition, epitaxial growth, and combinations thereof.

21. The method of claim 19 wherein the graft member comprises a solvatable polymer.

22. The method of claim 20 wherein the solvatable polymer comprising the coating and the solvatable polymer comprising the graft member are the same solvatable polymer.

23. The method of claim 19 wherein conjoining the solvatable polymer coating with the graft member using the solvent further comprises at least partially solvating both the coating and the graft member using the solvent, and allowing the solvated coating material and solvated graft member material to meld into a unitary structure.

24. The method of claim 19 wherein the graft member comprises a porous material different from the material comprising the solvatable polymer coating.

25. The method of claim 24 wherein conjoining the solvatable polymer coating with the graft member using the solvent further comprises at least partially solvating the coating using the solvent, and allowing the solvated coating material to flow into the pores of the graft member, thereby adhering the graft member to the coating.

26. A system for producing a stent-graft assembly, comprising:
means for providing a stent framework;
means for applying a solvatable polymer coating to the stent framework;
means for placing a generally cylindrical graft member adjacent to the solvatable polymer coating;
means for subjecting the solvatable polymer coating and adjacent graft member to a solvent;
means for conjoining the solvatable polymer coating with the graft member using the resulting solvate; and
means for solidifying the conjoined solvatable polymer coating and graft member.
